(19) United States
(12) Patent Application Publication
(10) Pub. No.: US 2004/0086932 A1
(43) Pub. Date: May 6, 2004

(54) Universal gel and methods for use thereof

(76) Inventors: T. Christian Boles, Lexington, MA (US); Lawrence Weir, Hopkinton, MA (US); Benjamin B. Stone, Holliston, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/721,157

(22) Filed: Nov. 25, 2003

Related U.S. Application Data
(63) Continuation of application No. 10/024,944, filed on Dec. 19, 2001.
Application No. 10/024,944 is a continuation of application No. 09/336,609, filed on Jun. 18, 1999, now abandoned.
(60) Provisional application No. 60/090,063, filed on Jun. 19, 1998.

(51) Int. Cl. C12Q 1/68

(52) U.S. Cl. 435/6

(57) ABSTRACT

The present invention provides rapid detection methods that detect virtually all non-viral organisms, from a broad to a narrow spectrum. More particularly, the present invention provides SRP RNA nucleic acid probes and methods of using such nucleic acid probes for the rapid, sensitive detection of non-viral organisms such as bacteria, fungi, and protozoa. Using the detection methods of the present invention, major non-viral groups such as bacterial, fungi, and protozoa, as well as specific species, can be identified in samples. In addition, kits for use in carrying out the methods of the present invention are provided.

### CROSS REFERENCE TO RELATED APPLICATIONS

The present application claims priority to U.S. patent application Ser. No. 60/090,063, filed Jun. 19, 1998 and is related to U.S. patent application Ser. No. 08/971,845, filed Aug. 8, 1997, herein both incorporated by reference.

### GOVERNMENT RIGHTS

Not applicable.

### BACKGROUND OF THE INVENTION

Bacterial, protozoan and fungal infections have increased in recent years due to an increasing population of immunocompromised patients, intensive immunosuppressive chemotherapy, and widespread use of broad-spectrum antibiotics and central venous catheters (Beck-Sague, et al., J. Inf. Dis., 167:1247-1251 (1993)). In addition, infection of medical supplies that are administered to patients, such as whole blood, plasma, platelets, packed red blood cells, bone marrow, lymphocytes, and serum, presents a critical problem.

Standard methods for diagnosis of such infections include culture and histopathology; however, these methods have limited sensitivity and specificity (see, e.g., Duthie et al., Clin. Inf. Dis. 20:598-605 (1995); Kahn et al., Am. J. Clin. Path. 86:518-523 (1986); and Thaler et al., Ann. Int. Med. 108:88-100 (1998)). A nucleic acid-based assay for the detection of non-viral nucleic acids may be an optimal diagnostic approach because it offers the potential of 1) higher sensitivity than current culture-based methods, and 2) applicability to multiple organisms, from a broad to a narrow spectrum.

Efforts have been made to detect organisms using specific and “universal” probes targeted to the multi-copy ribosomal RNA (see, e.g., U.S. Pat. No. 4,851,330; U.S. Pat. No. 5,714,324; U.S. Pat. No. 5,288,611; U.S. Pat. No. 5,688,645; U.S. Pat. No. 5,714,321; U.S. Pat. No. 5,693,469; U.S. Pat. No. 5,693,468; U.S. Pat. No. 5,691,149; U.S. Pat. No. 5,683,876; U.S. Pat. No. 5,681,698; U.S. Pat. No. 5,679,520; U.S. Pat. No. 5,677,129; U.S. Pat. No. 5,677,128; U.S. Pat. No. 5,677,127; U.S. Pat. No. 5,674,684; U.S. Pat. No. 5,595,874; U.S. Pat. No. 5,593,841; U.S. Pat. No. 5,547,842; U.S. Pat. No. 5,541,308; U.S. Pat. No. 5,216,143; U.S. Pat. No. 5,567,587; and U.S. Pat. No. 5,601,984).

However, alternative approaches are needed to provide a full range of broad and narrow spectrum detection capabilities. In view of the foregoing, there remains a need in the art for rapid, sensitive and highly specific methods for the detection of non-viral organisms such as fungi, protozoa and bacteria. The present invention remedies this and other needs.

### SUMMARY OF THE INVENTION

It has now been discovered that a nucleic acid probe to a signal recognition particle RNA can be used in an assay for rapid, specific detection of virtually all non-viral organisms. The assay provides both universal and specific probes that have the ability to detect a broad or a narrow spectrum of non-viral organisms, as desired. As such, the present invention provides probes and methods of using such probes to specifically detect virtually any phylogenetic grouping of organisms, e,g., from a kingdom such as eubacteria to a species such as E. coli, without cross-reactivity to members of other phylogenetic groups. Thus, using the methods of the present invention, non-viral infections can be diagnosed prior to other clinical or laboratory indications of invasive non-viral infections.

Generally, in the methods of the present invention, a non-viral organism that belongs to a group, e.g., a phylogenetic classification such as a kingdom, order, or species, is detected in a sample. The sample, which comprises SRP RNA, is contacted with a nucleic acid probe that is substantially complementary to a subsequence of SRP RNA from the group of non-viral organisms to be detected. The nucleic acid probe is hybridized to the sample under hybridization conditions such that the nucleic acid probe hybridizes to SRP RNA from the desired group of non-viral organism, but does not detectably hybridize to SRP RNA from other groups of non-viral organisms. The non-viral organism is detected by detecting hybridization of the nucleic acid probe to the SRP RNA.

As such, in one embodiment, the present invention provides a method for detecting the presence of a non-viral organism such as a bacteria in a sample, the method comprising: (a) contacting a sample comprising SRP RNA with a nucleic acid probe that is substantially complementary to a subsequence of SRP from the specified group of non-viral organisms and has the ability to hybridize under stringent conditions to the SRP from the group of non-viral organisms; (b) incubating the sample under stringent hybridization conditions to form a duplex SRP RNA, where the nucleic acid probe has hybridized to the SRP RNA; (c) contacting the duplex SRP RNA with a gel-immobilized nucleic acid probe, where the gel immobilized nucleic acid probe is substantially complementary to a subsequence from either the SRP RNA or the nucleic acid probe, which form the duplex SRP RNA; (d) incubating the duplex SRP RNA and the gel-immobilized nucleic acid probe under hybridization conditions such that the gel-immobilized nucleic acid probe hybridizes to a subsequence of either the SRP RNA or the nucleic acid probe, but does not detectably hybridize to SRP RNA from other non-viral organisms that do not belong to the group; and

(e) detecting hybridization of the gel-immobilized probe to duplex SRP RNA.

In another embodiment, more than one nucleic acid probe, which is substantially complementary to SRP RNA from a specified group of non-viral organisms, is used to hybridize under stringent conditions to the SRP RNA. In another embodiment, the nucleic acid probe or the SRP RNA comprise a detectable moiety. In another embodiment, the nucleic acid probe is about 8 to about 50 nucleotides in length, preferably about 15-25 nucleotides in length. In another embodiment, the nucleic acid probe is selected from the group consisting of DNA, peptide nucleic acid, and 2′-O-methyl RNA. In another embodiment, the nucleic acid probe is perfectly complementary to the subsequence of SRP RNA.

In another embodiment, the SRP RNA is 4.5S or 7S RNA.

In another embodiment, the sample is from a human.

In another embodiment, the non-viral organism is a bacterium, a fungus, or a protozoan.

In another embodiment, the SRP RNA sample is electrophoresed through a gel in which a nucleic acid probe has been immobilized.

In a further embodiment, the present invention provides kits for use in carrying out the methods of the present invention.

Other features, objects and advantages of the invention and its preferred embodiments will become apparent from the detailed description which follows.

### BRIEF DESCRIPTION OF THE DRAWINGS

FIG. 1 shows a northern blot of seven bacterial species probed with a conserved 22-mer from 4.5S SRP RNA.

FIG. 2 shows a northern blot comparing the abundance of 5S rRNA and 4.5S SRP RNA.

FIG. 3 shows detection of 4.5S SRP RNA in E. coli using capture with a gel-immobilized probe and adaptor, and detection with a fluorescent sandwich probe.

FIG. 4 shows a schematic of capture and detection using an in gel strand displacement technique.

FIG. 5 shows a schematic of capture with an adaptor.

FIG. 6 shows a schematic of gel-based capture.

FIG. 7 shows a northern blot of nine bacterial species probed with a pool of five gel-immobilized probes from 4.5S SRP RNA.

### DETAILED DESCRIPTION OF THE INVENTION AND PREFERRED EMBODIMENTS

I. Introduction

The present invention provides rapid detection methods that detect virtually all phylogenetic groups of non-viral organisms, without cross-reactivity to other groups. As previously explained, it has now been discovered that nucleic acid probes to SRP RNA can be used in an assay to detect both a broad and a narrow spectrum of non-viral organisms. As such, the present invention provides the ability to detect groups of non-viral organisms ranging from members of a kingdom, e.g., eubacteria, to members of a specific species, e.g., E. coli.

Signal recognition particle RNA, or SRP RNA, provides an ideal target for detection of a broad or narrow spectrum of non-viral organisms. SRP RNA is found in all non-viral organisms, from prokaryotes to higher eukaryotes (see, e.g., WO 97/03197).

Furthermore, SRP RNA has regions that are conserved in phylogenetic groups as large as, e.g., bacteria. Probes can therefore be designed that specifically hybridize to all members of a desired phylogenetic group, but not to other organisms outside of the specified group. Alternatively, probes that hybridize to genera or species specific regions can also be designed. The methods of the invention therefore have wide application to detection of a broad to a narrow spectrum of organisms. Moreover, SRP RNA is present in high copy number is cells (e.g., 2000 copies per cell in E. coli), making it an extremely useful target for detection.

Rapid detection of non-viral organisms is useful for a number of applications, including human and veterinary diagnostics, screening medical and food supplies, screening for soil and water contamination, and agricultural uses. In one embodiment, the methods of the invention are useful for detecting a broad spectrum of bacteria that are potential contaminants of medical supplies such as whole blood, platelets, plasma, lymphocytes, packed red blood cells, serum, bone marrow and the like. The methods of the invention are also useful for diagnosing infection with specific organisms, e.g., bacterial sepsis caused by Serratia marcescens, Staphylococcus epidermidis, Staphylococcus aureus, Pseudomonas aeruginosa, Escherichia coli, Bacillus cereus, Enterobacter cloacae, and Streptococcus pyogenes; opportunistic fungal infections caused by Candida albicans, Aspergillus flavus, and Cryptococcus neoformans; and protozoan diseases such as malaria, Chagas disease, sleeping sickness, and the like, caused by Plasmodium falciparum, Leishmania brucei, and Trypanosoma cruzi.

The methods of the present invention can be used to identify members of broad groups such as mammals, vertebrates, invertebrates, bacteria, fungi, and protozoa, as well as distinguishing between specific genera or species of such groups. Preferred groups of non-viral organisms for detection include bacteria, fungi, and protozoa. Particularly preferred genera for detection include bacterial genera, such as Propionibacterium sp., Klebsiella sp., Enterobacter sp., Serratia sp., Salmonella sp., Legionella sp., Pseudomonas sp., Haemophilus sp., Escherichia sp., Mycoplasma sp., Micrococcus sp., Listeria sp., Bacillus sp., Staphylococcus sp., Streptococcus sp., Clostridia sp., Neisseria sp., Helicobacter sp., Vibrio sp., Campylobacter sp., Bordetella sp., Ureaplasma sp., Treponema sp., Leptospira sp., Borrelia sp., Actinomyces sp., Nocardia sp., Chlamydia sp., Rickettsia sp., Coxiella sp., Ehrilichia sp., Rochalimaea sp., Brucella sp., Yersinia sp., Fracisella sp., and Pasteurella sp; fungal genera such as Candida sp., Cryptococcus sp., Aspergillus sp., Histoplasma sp., and Microsporum sp.; and protozoa genera such as Pneumocystis sp., Toxoplasma sp., Cryptosporidium sp., Giardia sp., Leshmania sp., Trypanosoma sp., Plasmodium sp., Acanthamoeba sp., and Entamoeba sp.

Preferred bacterial species for detection include Serratia marcescens, Staphylococcus epidermidis, Staphylococcus aureus, Pseudomonas aeruginosa, Escherichia coli, Bacillus cereus, Enterobacter cloacae, Streptococcus pyogenes, Staphylococcus warneri, Streptococcus (à-hemolytic), Streptococcus pneumoniae, Streptococcus mitis, Serratia liquifaciens, Propionibacterium acnes, Yersinia enterocolitica, Pseudomonas fluorescens, and Pseudomonas putida.

In the method of the invention, an SRP RNA probe is designed that has the ability to specifically detect a group of organisms, such as bacteria, or a specific species. A sample suspected of having the specified non-viral organism is incubated with the probe. The probe hybridizes to SRP RNA from the group of choice, but does not detectably hybridize to SRP RNA from other non-viral organisms not in the group. The non-viral organism is detected by detecting hybridization of the probe to the SRP RNA, e.g., with a direct or indirect detectable moiety.

In one embodiment, the sample comprising SRP RNA is incubated with a nucleic acid probe that hybridizes to the SRP RNA of choice, forming a duplex SRP RNA. The duplex SRP RNA is contacted with a gel-immobilized probe, which is substantially complementary to either the nucleic acid probe or the SRP RNA in the duplex. Alternatively, the SRP RNA is contacted first with the gel-immobilized probe, forming a duplex SRP RNA. The duplex is then contacted with a nucleic acid probe that is substantially complementary to the SRP RNA. The nucleic acid probe or the SRP RNA are preferably labeled, either directly or indirectly, with a detectable moiety. In a preferred embodiment, the SRP RNA is electrophoresed through a gel, where it is captured by the gel-immobilized probe. The nucleic acid probe is added to the SRP RNA before or after to electrophoresis. Optionally, an adaptor probe is used, which hybridizes to both the SRP RNA and the gel-immobilized probe, and a third labeled probe is used for detection of the SRP RNA.

As generally described above, the detection of non-viral organisms using the methods of the present invention requires multiple steps. These steps, which will be explained in greater detail hereinbelow, generally include designing and making broad and narrow spectrum probes, preparing the sample by lysing cells to release target nucleic acid, hybridizing the probes to the target SRP sequences, and detecting the hybridized sequences.

II. Definitions

An “SRP RNA” refers to the RNA component of a ribonucleoprotein signal recognition particle. SRP RNA refers to both the mammalian or eukaryotic 7S or 7SL RNA as well as the 4.5S or scRNA RNA in prokaryotes (see, e.g., Ribes et al., Cell 63:591-600 (1990); Nakamura et al., J. Bacteriol. 174:2185-2192 (1992); and Poritz et al., Science 250:111-1117 (1990)).

A “non-viral organism” refers to any organism except viruses.

A “group” refers to a phylogenetic relationship among organisms, e.g., kingdom, phylum, class, order, family, genus, species, or strain or sub-type. A “group consisting of at least one but less than all non-viral organisms” describes the smallest to the largest group of non-viral organisms that are detected by the assays of the invention. The “group consisting of at least one non-viral organism” refers to a small set of organisms, e.g., a species, sub-type, or strain, and the ability to distinguish between small groups, e.g., the ability to specifically detect E. coli. The group consisting of “less than all non-viral organisms” describes a large set of organisms, e.g., from a kingdom or phylum, e.g., eubacteria or bacteria, and the ability to distinguish between large groups, e.g., the ability to specifically detect bacteria.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. For example, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide. Where a specific nucleic acid sequence is given, it is understood that the complementary strand is also identified and included as the complementary strand will work equally well in situations where the target is a double stranded nucleic acid.

A “detectable moiety” is a composition detectable by spectroscopic, photochemical, biochemical, immunochemical, or chemical means. For example, useful detectable moieties or labels include, but are not limited to, ³²P, fluorescent dyes, electron-dense reagents, enzymes (e.g., as commonly used in an ELISA), biotin, digoxigenin, or haptens and proteins for which antisera or monoclonal antibodies are available.

“Nucleic acid probe” or “probe” refers to an oligonucleotide that binds through complementary base pairing to a subsequence of a target nucleic acid. The nucleic acid probe may be, for example, a DNA fragment prepared by amplification methods such as by PCR or, it may be synthesized by either the phosphoramidite method described by Beaucage and Carruthers (Tetrahedron Lett., 22:1859-1862 (1981)), or by the triester method according to Matteucci, et al. (J. Am. Chem. Soc., 103:3185 (1981)), both of which are incorporated herein by reference. By assaying for the presence or absence of the probe, one can detect the presence or absence of the select sequence or subsequence.

A “labeled nucleic acid probe or oligonucleotide” is one that is bound, either covalently, through a linker or a chemical bond, or noncovalently, through ionic, van der Waals, electrostatic, or hydrogen bonds to a detectable moiety such that the presence of the probe may be detected by detecting the presence of the detectable moiety bound to the probe.

Probes are optionally directly labeled with detectable moieties, for example, radioisotopes and fluorescent molecules, or indirectly labeled with, for example, biotin or digoxigenin which are used in conjunction with their labeled, naturally occurring anti-ligands.

“Gel-immobilized nucleic acid probes” are nucleic acid probes that are covalently bound to an electrophoresis medium, such as paper, polymers such as agarose and acrylamide, and the like, or are covalently bound to particles suspended in the electrophoresis matrix so that they do not migrate under the influence of an applied electrical field (see, e.g., U.S. patent application Ser. No. 08/971,845, filed Aug. 8, 1997, herein incorporated by reference).

An “adaptor” probe is a nucleic acid probe that has regions of substantial complementarity to both the SRP RNA and a gel-immobilized probe. The adaptor has the ability to hybridize to both the SRP RNA and the gel-immobilized probe at the same time in different regions, indirectly linking the SRP RNA and the gel-immobilized probe.

“Duplex SRP RNA” refers to an SRP RNA to which a nucleic acid probe has hybridized, forming a duplex nucleic acid with a subsequence of the SRP RNA. A gel-immobilized probe that is substantially complementary to a duplex SRP RNA has the ability to hybridize either to a subsequence of the SRP RNA not already part of a duplex, or to a subsequence of the nucleic acid probe that is not already part of the duplex.

The term “substantially complementary” refers to a nucleic acid segment that will hybridize, under stringent hybridization conditions, to a complement of another nucleic acid strand. As is known to one of skill in the art, stringent hybridization conditions can be adjusted within the designated range to allow for higher or lower percent mismatches between a probe and its target.

The term “perfectly complementary” refers to a nucleic acid that has no mismatches when hybridized to its complementary nucleic acid strand, e.g., the complement of the complement has 100% identity with the target nucleic acid subsequence. A perfectly complementary probe is also substantially complementary.

“Subsequence” refers to a sequence of nucleic acids which comprise a part of a longer sequence of nucleic acids.

“Hybridizing” refers the binding of two single stranded nucleic acids via complementary base pairing. “Selective hybridization” refers to the binding, duplexing, or hybridizing of a molecule only to a particular nucleotide sequence under stringent hybridization conditions when that sequence is present in a complex mixture (e.g., total cellular or library DNA or RNA). A nucleic acid probe of the invention is substantially complementary to SRP RNA and selectively hybridizes under stringent conditions to SRP RNA from the group of choice, but not to SRP RNA from organisms outside the group of choice. For selective or specific hybridization, a positive signal is at least two times background, preferably 10 times background hybridization.

“Hybridization conditions such that the nucleic acid probe hybridizes to SRP RNA from the group of non-viral organisms, but does not detectably hybridize to SRP RNA from other non-viral organisms that do not belong to the group” refers to selective hybridization as defined herein.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 600° C. for long probes (e.g., greater than 50 nucleotides). Stringent conditions may also be achieved with the addition of destabilizing agents such as formamide. For stringent hybridization, a positive signal is at least two times background, preferably 10 times background hybridization. Exemplary high stringency hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

The term “sample” as used herein refers to food, clinical, medical, and environmental samples suspected of containing a non-viral organism, or those samples used as a control in an assay for a non-viral organisms. Clinical samples include, but are not limited to, the following: blood, urine, cerebrospinal fluid, skin and/or other tissue biopsies, saliva, synovial fluid, sputum, bronchial wash, bronchial lavage, and other tissue or fluid samples from human patients or veterinary subjects. Medical supply samples include whole blood, platelets, plasma, packed red blood cells, lymphocytes, bone marrow, serum and the like. Food samples include, but are not limited to, the following: meats, dairy products, beverages, grains, nuts, fruits, juices and vegetables, all of which may be cooked, partially cooked or uncooked. Environmental samples include soil, water, and vegetation samples. Samples can be from humans, mammals, plants, and the like.

The terms “identical” or percent “identity,” in the context of two or more nucleic acids sequences, refer to two or more sequences or subsequences that are the same or have a specified percentage of nucleotides that are the same, when compared and aligned for maximum correspondence over a comparison window, as measured using one of the following sequence comparison algorithms or by manual alignment and visual inspection.

The phrase “substantially identical,” in the context of two nucleic acids refers to sequences or subsequences that have at least 70%, preferably 85%, most preferably 90-95% nucleotide identity when aligned for maximum correspondence over a comparison window as measured using one of the following sequence comparison algorithms or by manual alignment and visual inspection. This definition also refers to the complement of a test sequence, which has “substantial complementarity” when the test sequence has substantial identity to a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A comparison window and sequence algorithm programs are typically used to make the comparison, as described below.

III. Design and Preparation of Nucleic Acid Probes

Probes used in the methods of the invention are derived from and complementary to SRP RNA sequences. For probes that recognize individual species, typically a probe of suitable size is designed from the SRP RNA from that species. For probes that recognize larger groups of organisms, e.g., bacteria, fungi, protozoa, SRP RNAs from a variety of organisms within the desired group are compared for regions of sequence conservation. A probe is then designed that is substantially complementary to the members of the group, but not to other organisms outside the group.

Probes suitable for use in the methods of the present invention can be identified using sequence analysis techniques known to those of skill in the art. Sequence analysis programs can be used to compare SRP RNAs from different organisms and identify regions of substantial complementarity. When using a sequence comparison algorithm, sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and is visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps. PILEUP can be obtained from the GCG sequence analysis software package, e.g, version 7.0 (Devereaux et al., Nuc. Acids Res. 12:387-395 (1984).

Another example of algorithms that are suitable for determining percent sequence identity and sequence similarity are the BLAST and the BLAST 2.0 algorithm, which are described in Altschul et al., J. Mol. Biol. 215:403-410 (1990) and Atschul et al., Nucleic Acids Res. 25:3389-3402 (1977)). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, alignments (B) of 50, expectation (E) of 10, M=5, N=4, and a comparison of both strands. The BLASTP program (for amino acid sequences) uses as defaults a wordlength (W) of 3, and expectation (E) of 10, and the BLOSUM62 scoring matrix (see Henikoff& Henikoff, Proc. Natl. Acad. Sci. USA 89:10915 (1989)).

In one embodiment, a 22-mer sequence represented by nucleotides 44-65 of E. coli 4.5S RNA is conserved across bacteria (E. coli sequence GUCAGGUCCGGAAGGAAGCAG; (SEQ ID NO: 1)). The complement of this region thus provides a preferred probe for detection of bacteria: GCTGCTTCCTTCCGGACCTGAC (SEQ ID NO:2). Four shorter probes derived from this region are also preferred for identification of bacteria: GCTGCTTCCTTCCGGACCTGA (SEQ ID NO:3); GCTGCTTCCTTC (SEQ ID NO:4); GCTGCTTCCTTCCG (SEQ ID NO:5), GACCTGACCTGGTA (SEQ ID NO:6). Probes from this conserved region that act as adaptor probes can also be used GCTGCTTCCTTCCGGACCTGAGTGAATACGTTCCCGGGCCT (SEQ ID NO:7); and GCTGCTTCCTTCCGGACCTGACAAAAACGATAAACCAACCA (SEQ ID NO:8). A probe suitable for detection of E. coli species is GGCACACGCGTCATCTGC (SEQ ID NO:9).

In another embodiment, a 30mer sequence represented by nucleotides 36-65 of E. coli 4.5S RNA (GenBank accession number X01074) is conserved across bacteria (E. coli sequence: UUUACCAGGUCAGGUCCGGAAGGAAGCAG: SEQ ID NO:10). The complement of this region thus provides a preferred probe for detection of bacteria: GCTGCTTCCTTCCGGACCTGACCTGGTAAA (SEQ ID NO:11).

Sequences for SRP RNA can be obtained through publicly available databases, e.g., on the world wide web at http://www.medkem.gu.se/dbs/SRPDB/, or GenBank database. An alignment of SRP RNAs is also found in Larsen & Zweib, Nuc. Acids Res. 24:80-81 (1996). Alternatively, if a specific SRP sequence is desired, it can be cloned and sequenced using standard techniques known to those of skill in the art. Related, known SRP RNA sequences can be used as probes to identify such sequences in cDNA libraries, or to make primers for amplification of such sequences. In one embodiment, the SRP is first purified, the SRP RNA extracted, reverse transcribed, cloned, and sequenced. If desired, the sequences are aligned using the sequence comparison algorithms described above (see, e.g., the Wisconsin Sequence Analysis Package (Genetics Computer Group, Madison, Wis.) (Devereux, et al., Nucleic Acids Research 12:387-395 (1984)). Conserved regions are typically compared using sequence comparison algorithms to sequences outside the desired group to provide substantial complementarity within the group but not outside the group. Probes can also be tested using hybridization methods known to those of skill in the art and described below to identify probes that are substantially complementary to members of the desired group, but not to members outside the group. The probes are then optimized for melting temperature (Tₘ) equivalence, lack of duplex, hairpin or primer dimer formation, and internal stability (see, e.g., OLIGO software, National Biosciences Inc., Plymouth, Minn.).

Probes are typically synthesized chemically according to the solid phase phosphoramidite triester method described by Beaucage and Caruthers, Tetrahedron Letts. 22(20):1859-1862 (1981), e.g., using an automated synthesizer, e.g., as described in Needham-VanDeventer, et al., Nucleic Acids Res. 12:6159-6168 (1984) (“NeedhamVanDeventer”). Moreover, oligonucleotides can also be custom made and ordered from a variety of commercial sources known to persons of skill in the art. Purification of oligonucleotides, where necessary, is typically performed by either native acrylamide gel electrophoresis, or by anion-exchange HPLC as described in Pearson & Regnier, J Chrom. 255:137-149 (1983). The sequence of the synthetic oligonucleotides can be verified using the chemical degradation method of Maxam and Gilbert in Methods in Enzymology (Grossman & Moldave, eds., 1980)).

The nucleic acid probes of the invention are typically 8-50 nucleotides long, preferably 15-25 nucleotides long. The nucleic acid probes of the invention preferably include DNA probes, PNA probes and 2′-O-methyl ribonucleotide probes.

The nucleic acid probes of the invention include probes labeled with detectable moieties for detection of hybridization, gel-immobilized probes, and adaptor probes, as described below. Nucleic acid probes can be used to capture and to detect the SRP RNA of choice in either solution or in solid phase. For example, nucleic acid probes can be immobilized on a solid surface, such as a bead, a plate, a dipstick, etc. In one embodiment, gel-immobilized probes can be used to directly capture the SRP RNA of choice. The gel-immobilized probes are synthesized as described below, and then polymerized in a gel via covalent attachment to a polymer that forms a gel, e.g., agarose or acrylamide, or via covalent attachment to a particle suspended in the gel (for methods of gel-immobilized probe synthesis, covalent attachment to polymers, and polymerization of gels containing gel-immobilized probes, see U.S. patent application Ser. No. 08/971,845, filed Aug. 8, 1997, herein incorporated by reference).

Any suitable method can be used to bind the gel-immobilized probe to the starting electrophoresis material. Probes can be coupled to gel materials using, e.g, thiol-reactive groups, carboxyl groups, primary amine groups and the like (see, e.g., U.S. patent application Ser. No. 08/971,845 and U.S. patent application Ser. No. 08/812,105, herein incorporated by reference). For example, DNA gel-immobilized probes can be made by automated synthesis using a phosphoramidite to which a polymerizable ethylene group has been added. This phosphoramidite with a polymerizable ethylene group is known as Acrydite™ and is commercially available from Mosaic Technologies (Boston, Mass.). Gel-immobilized probes with any desired sequence can be made with this method. Gel-immobilized probes include nucleic acids as defined above (e.g., DNA, RNA, PNA, 2-O-methyl RNA, etc.). Gel-immobilized nucleic acid probes can be used with any standard electrophoretic technique, e.g., slab or tube gels made from gel-forming polymers, such as agarose and cross-linked acrylamide; capillary electrophoresis using non-gel forming polymers such as linear polyacrylamide; paper electrophoresis; and the like.

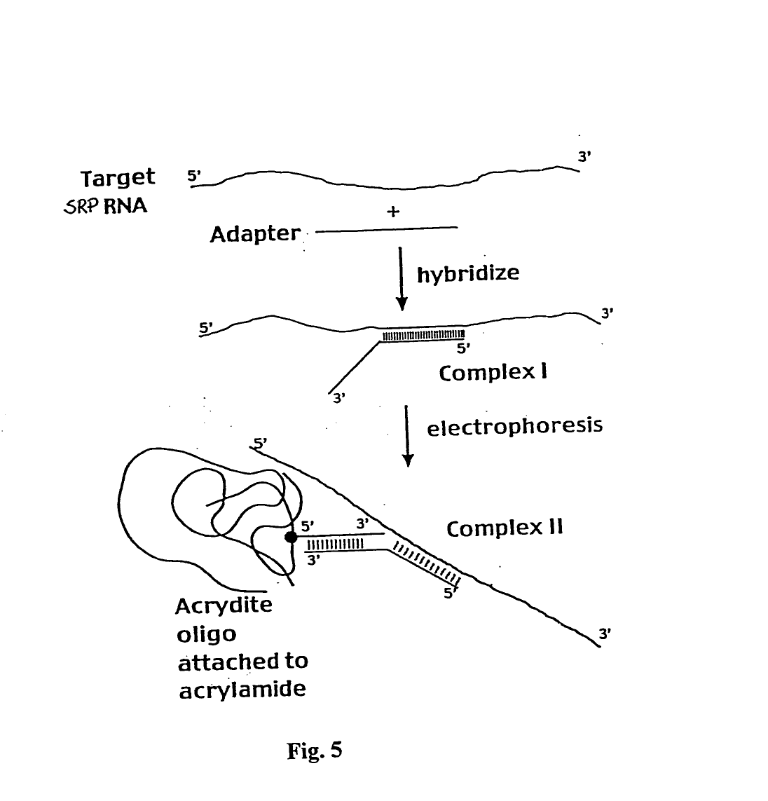

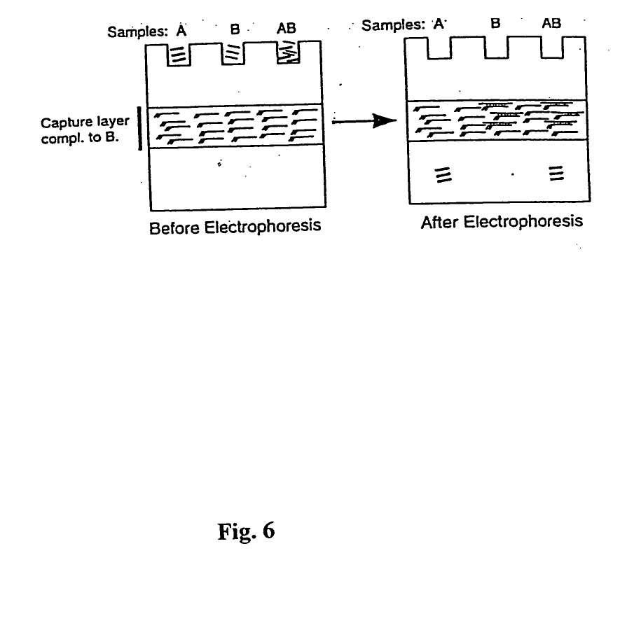

Gel-immobilized probes can directly capture SRP RNA (see FIG. 6), or they can indirectly capture SRP RNA through the use of an adaptor probe (see FIG. 5). Adaptor probes have regions of substantial complementarity to both the gel-immobilized probe and the SRP RNA, and are used to link the gel-immobilized probe and the SRP RNA through hybridization to both molecules at the same time.

Nucleic acid probes labeled with detectable moieties can be used to detect hybridization of the probes to SRP RNA. Nucleic acid probes labeled with detectable moieties are substantially complementary to any suitable region of the SRP RNA, e.g., a subsequence other than the one that is complementary to the gel-immobilized probe or the adaptor probe. Probes are labeled with detectable moieties as described herein.

Typically, all probe sequences are selected to hybridize only to a substantially or perfectly complementary RNA or DNA. The probes are selected so that little or no secondary structure forms within the probe. Self-complementary probes have poor hybridization properties because the complementary portions of the probes form hairpin structures. The probes are also selected so that the probes do not hybridize to each other, thereby preventing duplex formation of the probe and target.

IV. Sample Preparation

The sample used in the methods of the present invention can be obtained from any source, i.e., food, medical supplies, and clinical and environmental sources, suspected of containing a non-viral organism. Clinical samples include, for example, blood, urine, cerebrospinal fluid, skin and/or other tissue biopsies, saliva, synovial fluid, sputum, bronchial wash, bronchial lavage, and other tissue or fluid samples from human patients or veterinary subjects. Food samples include, for example, meats, dairy products, beverages, grains, nuts, fruits, juices and vegetables, all of which may be cooked, partially cooked or uncooked. Medical supply samples include products that are administered to patients such as whole blood, platelets, plasma, bone marrow, lymphocytes, and serum. Environmental samples include public or private water supplies, soil, vegetation, water from lakes, rivers, and oceans, and the like.

The cell wall and/or membrane of the non-viral organisms must be efficiently lysed or disrupted in a manner that releases the target SRP RNA so it is available for hybridization with the nucleic acid probe. The target SRP RNA is then treated or recovered from such cells so as to be sufficiently free of potentially interfering substances, such as enzymes, in particular ribonuclease, or other components that might interfere with hybridization of a probe to the target nucleic acid sequences.

As the target nucleic acid is SRP RNA, care must be taken to avoid degradation of the RNA during sample preparation and hybridization. A number of reagents are useful for releasing intact RNA from the cells in the test sample. Each of these reagents lyses cells in the sample and concomitantly minimizes or eliminates nuclease activity during the RNA isolation procedure by denaturing or digesting proteins, including ribonucleases: guanidine hydrochloride; guanidine isothiocyanate; sodium dodecyl sulfate or sarcosyl and proteinase K or pronase (see, e.g., Farrell, RNA Methodologies (1993)). In addition, specific nuclease inhibitors can be added to the sample, such as RNase inhibitors, e.g., placental RNase inhibitor enzyme (Blackburn, J. Biol. Chem. 254:12484 (1970)) and vanadyl-ribonucleoside complexes.

Standard laboratory techniques can be used to lyse cells and release RNA from samples (see, e.g., Sambrook et al., supra; Ausubel et al., supra). One preferred method of isolating RNA from cells is based on the method of Chomczynski, Biotechniques 15:532-535 (1993)). This method uses a single reagent for isolation of RNA, or RNA and DNA. First, the cells are lysed using a guanidine isothiocyanate-phenol buffer. The sample is homogenized in this buffer and then separated into aqueous and organic phases by addition of chloroform and centrifugation. The RNA is then precipitated from the aqueous phase and resuspended in RNase free solution. DNA can also be isolated along with RNA using this technique. Kits based on this method are commercially available, e.g., the TRI Reagent® (Molecular Research Center, Inc.). A variety of other techniques can also be used to isolate RNA, such as those that use SDS/proteinase K treatment followed by extraction with a phenol solution. RNA can be further purified using centrifugation with a cesium chloride gradient. RNA can also be isolated using the silica gel binding/anion exchange method. DNA can be removed from the RNA by treating with RNase free DNase.

A number of additional techniques well known to those of skill in the art can be used to lyse or disrupt the non-viral cell wall and/or membrane. Mechanical lysis is one such technique and can be achieved by sonication, or by multiple freeze/thaw cycles. Glass beads or enzymatic methods of cell wall and membrane disruption are also preferred. In addition, chemical means of cell disruption can be used and include standard lysing means such as lysozymes and osmotic shock.

Enzymatic methods typically allow consistent release of nucleic acid from samples of small quantity, where physical contact for disruption cannot be assured. In a one embodiment, lyticase treatment (Sigma, St. Louis, Mo.) is used to disrupt the cell wall. Snail gut enzyme is the prototype enzyme used for cell wall lysis, but the preparation can have some variability in activity from batch to batch (Kitamura, et al., Journal of General Applied Microbiology 18:57-71 (1972); Kitamura, et al., Journal of General Applied Microbiology 20:323-344 (1974)). â-1,3-glucanase enzymes hydrolyze glucose polymers at â-1,3-glucan linkages to release laminaryipentaose and result in spheroplasts, modified organisms with partial loss of the cell wall and increased osmotic sensitivity (Pringle, et al., Journal of Bacteriology, 140:289-293 (1979)). â-1,3-glucanase products available for use include, but are not limited to, zymolyase (ICN Biomedicals, Costa Mesa, Calif.) (Kitamura, et al., Archives of Biochemistry & Biophysics 153:403-406 (1972)), which is purified from a submerged culture of Arthrobacter luteus in the fermentation of yeast, and lyticase (Sigma, St. Louis, Mo.) (Scott, et al., Journal of Bacteriology 142:414-423 (1980)), which is a genetically engineered synthetic equivalent. Zymolyase is an impure product; other enzymes found in the preparation include â-1,3-gluconase, protease, mannanase, amylase, xylanase, phosphatase, and trace DNAse. Use of a synthetic product avoids these impurities.

When the sample is a complex mixture, such as a food sample suspected of containing a fungal organism, it may be necessary to isolate the nucleic acid from the complex mixture, as described above. A variety of techniques for extracting nucleic acids from biological samples are known in the art. See, e.g., the extraction methods described by Higuchi in “Simple and Rapid Preparation of Samples for PCR” in PCR Technology (Erlich, ed., 1989)); Maniatis, et al., Molecular Cloning: A Laboratory Manual, (1982); Hagelberg & Sykes, Nature 342:485 (1989); and Arrand, Preparation of Nucleic Acid Probes in Nucleic Acid Hybridization, A Practical Approach (Haines & Higgins, eds., pp. 18-30 (1985)), all of which are incorporated herein by reference.

V. Hybridization Procedures

Once the sample is obtained, it is subjected to a nucleic acid hybridization protocol. Nucleic acid hybridization techniques suitable for hybridizing a nucleic acid probe to the target sequences using nucleic acid primers are well known to those of skill in the art (see, e.g., Ausubel, supra and Sambrook, supra). Choice of optimum temperatures and incubation times for the hybridization and/or electrophoresis of the specific target sequences of the invention can be determined by routine titration. Both solution phase and solid phase hybridization techniques can be used.

For example, in a standard solution phase hybridization, the nucleic acid probe is incubated with the sample comprising SRP RNA under hybridization conditions (temperature, time, buffer, target/probe concentration) that provide for specificity and low background (see, e.g., Examples 1-4). Hybridization solutions are well known to those of skill in the art, e.g., 5×SSC, 5× Denhardt's solution, 50% formamide and 1% SDS for use at a temperature of 42° C.; or 5×SSC, 5× Denhardt's solution and 1% SDS for use at a temperature of 68° C., and are also commercially available, e.g., Rapidhyb (Amersham). The time for hybridization can vary from about one hour to overnight. After hybridization in solution, the SRP duplex is isolated from the non-duplexed molecules, e.g., by digesting non-duplexed molecules and isolating the duplex via an affinity group such as a biotin molecule attached to the nucleic acid probe. Hybridization is then detected, typically with a second nucleic acid probe. Alternatively, the SRP RNA or the adaptor probe can be labeled with a detectable moiety (see detection methods, below).

For solid phase hybridization, a nucleic acid probe is bound to a solid substrate. For instance, the solid surface is optionally paper, or a membrane (e.g., nylon or nitrocellulose), a microtiter dish (e.g., PVC, polypropylene, or polystyrene), a test tube (glass or plastic), a dipstick (e.g., glass, PVC, polypropylene, polystyrene, latex, and the like), a microcentrifuge tube, a bead, or a glass, silica, plastic, metallic or polymer bead or other substrate as described herein. Preferably, the solid phase is a polymer that has the ability to form a gel, or a particle that has the ability to be suspended in a gel. The gel polymer can be agarose, acrylamide, and the like, at any suitable concentration, and the gel can be in any suitable form, e.g., a slab or a tube.

The nucleic acid probe can be covalently bound or noncovalently attached to the substrate through nonspecific binding. If covalent binding between a compound and the surface is desired, the surface will usually be polyfunctional or be capable of being polyfunctionalized. Functional groups that may be present on the surface and used for linking include, but are not limited to, carboxylic acids, aldehydes, amino groups, cyano groups, ethylenic groups, hydroxyl groups, mercapto groups and the like. In addition to covalent bonding, various methods for noncovalently binding a nucleic acid probe can be used (see, e.g., Essential Molecular Biology, (Brown, ed., 1993)); In Situ Hybridization Protocols (Choo, ed., 1994)). The hybridization reaction is performed as described above, and optionally, washes can be performed according to standard procedures. Detection is carried out as described below. A preferred method of covalently attaching an oligonucleotide to an acrylamide polymer via a polymerizable ethylene group is described in U.S. patent application Ser. No. 08/971,845, filed Aug. 8, 1997, herein incorporated by reference.

In a presently preferred embodiment, the SRP RNA is hybridized to both a fluorescently labeled nucleic acid probe and an adaptor probe (see Example 3).

Alternatively, the adaptor probe or the SRP RNA can be labeled with a detectable moiety, is dispensing with the need for a third probe (see FIG. 6). The adaptor probe has subsequences that are substantially complementary to both the SRP RNA and a gel-immobilized probe. The hybridization reaction is then loaded onto a gel, in which a probe has been immobilized (for a description of methods used to make and use gels containing gel-immobilized probes, see U.S. patent application Ser. No. 08/971,845, filed Aug. 8, 1997). Typically, the gel-immobilized probe is located in a discreet “capture zone” of the gel. For example, a 5% 29:1 acrylamide gel is polymerized in three layers, with a middle capture layer having the gel-immobilized probe at a concentration of 10 μM. The gel can be from about 4% acrylamide to about 20% acrylamide. The sample with SRP RNA is applied to the gel, which is run in a standard buffer, e.g., 0.5× TBE. Standard conditions are used to run the gel, e.g., 120 volts for about 1.5 hours. As the SRP RNA passes through the capture layer, the gel-immobilized probe captures the adaptor molecule (see FIG. 5 and FIG. 6). The SRP RNA is then detected by visualizing the fluorescent probe. Alternatively, the SRP RNA can be electrophoresed through the gel and directly hybridized to the capture probe, followed by electrophoresis and capture of a labeled probe. The preferred embodiment has very high signal to noise ratio, and can be used to detect perfectly complementary probe hybridization, compared to a target with one mismatch, with signal to noise rations of approximately 50-100 to 1.

VI. Detection of Hybridized Target SRP RNA

Detection methods using nucleic acid probes are well known to those of skill and a general review of such techniques can be found in Nucleic Acid Hybridization, A Practical Approach (Hames & Higgins, eds., 1985)). As such, no attempt to describe in detail each and every possible detection method will be made.

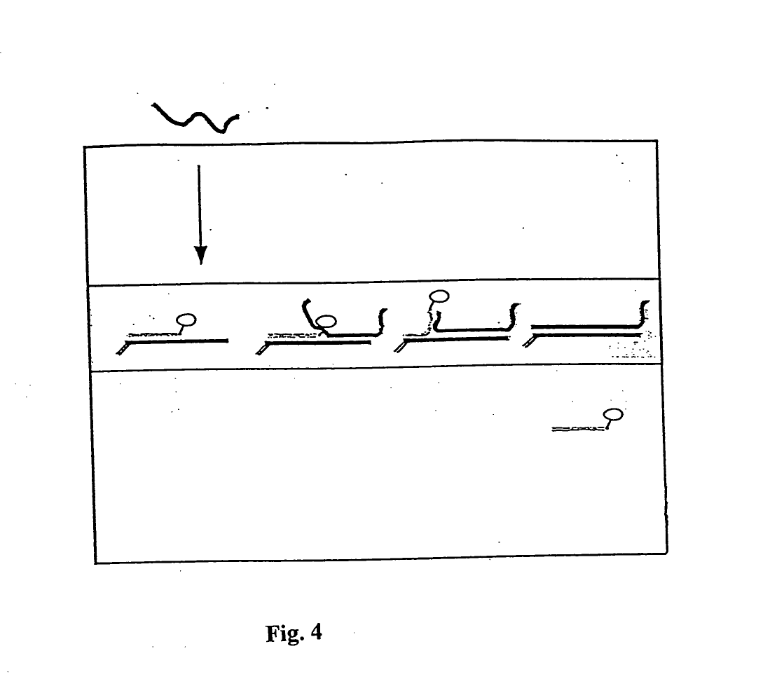

Both direct and indirect detection methods can be used in the present invention. For direct methods, a “sandwich” probe with a detectable moiety is used that directly binds to another subsequence of the captured SRP RNA. In another direct method, the SRP RNA itself is labeled with a detectable moiety. The adaptor probe can also be labeled with a detectable moiety. An indirect or competitive method of detection uses an in-gel strand displacement technique. In this technique, the gel-immobilized capture probe is hybridized to a labeled nucleic acid probe. When the SRP RNA binds to the capture probe, it displaces the labeled nucleic acid probe. Displaced, labeled nucleic acid probe is then detected (see FIG. 4).

Probes can be labeled with a detectable moiety by any one of several methods. The most common method of detection is the use of autoradiography with ³H, ¹²⁵I, ³⁵S, ¹⁴C, or ³²P labeled probes, or the like. The choice of radio-active isotope depends on preferences due to ease of synthesis, stability and half-lives of the selected isotopes. Other labels include, for example, ligands which bind to antibodies labeled with fluorophores, chemiluminescent agents, fluorescent agents, and enzymes. Alternatively, probes can be conjugated directly with labels such as fluorophores, chemiluminescent agents or enzymes. A wide variety of labels suitable for labeling nucleic acids and conjugation techniques are known and reported extensively in both the scientific and patent literature, and are generally applicable to the present invention for the labeling of nucleic acid probes for the detection of amplified target nucleic acid. The choice of label depends on sensitivity required, ease of conjugation with the probe, stability requirements, available instrumentation and disposal provisions.

The choice of label dictates the manner in which the label is bound to the probe. The probes can be labeled using radioactive nucleotides in which the isotope resides as a part of the nucleotide molecule, or in which the radioactive component is attached to the nucleotide via a terminal hydroxyl group that has been esterified to a radioactive component such as inorganic acids, e.g., ³²P phosphate or ¹⁴C organic acids or, esterified to provide a linking group to the label. Base analogs having nucleophilic linking groups, such as primary amino groups, can also be linked to a label.

Non-radioactive probes are often labeled by indirect means. For example, a ligand molecule is covalently bound to the probe. The ligand then binds to an anti-ligand molecule that is either inherently detectable or, covalently bound to a detectable signal system, such as an enzyme, a fluorophore or, a chemiluminescent compound. Ligands and anti-ligands may be varied widely. Where a ligand has a natural anti-ligand, namely ligands such as biotin, digoxigenin, thyroxine and cortisol, it can be used in conjunction with its labeled, naturally occurring anti-ligands. Alternatively, any haptenic or antigenic compound can be used in combination with antisera or an antibody.

Probes can also be labeled by direct conjugation with a label. For example, cloned DNA probes have been coupled directly to horseradish peroxidase or alkaline phosphatase (Renz & Kurz, Nuc. Acids Res. 12:3435-3444 (1984)), and synthetic oligonucleotides have been coupled directly with alkaline phosphatase (Jablonski, et al., Nuc. Acids. Res. 14:6115-6128 (1986)).

Enzymes of interest as labels are hydrolases, such as phosphatases, esterases and glycosidases or, oxidoreductases, particularly peroxidases. Fluorescent compounds include fluorescein and its derivatives, rhodamine and its derivatives, dansyl, umbelliferone, etc. Chemiluminescers include luciferin and 2,3-dihydrophthalazinediones, e.g., luminol.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like.

Similarly, enzymatic labels can be detected by providing appropriate substrates for the enzyme and detecting the resulting reaction product. Finally, simple colorimetric labels are often detected simply by observing the color associated with the label. Thus, in various dipstick assays, conjugated gold often appears pink, while various conjugated beads appear the color of the bead.

A preferred mode of detecting target sequences is hybridization to the target SRP RNA of a second probe with a detectable moiety. The second probe is substantially complementary to a subsequence of the target SRP RNA. Before or after hybridization to the nucleic acid probe, the target sequence can be captured on a solid support, such as nylon or nitrocellulose membrane, or a gel. The second probe can be radioactively tagged or attached directly or indirectly to an enzyme molecule. Then, either before or after capture of the target sequence, the target sequence is incubated with the second probe under hybridization conditions and excess probe is washed away. Detection can be by autoradiography, radiation counting or radioactive probe, by exposure to an antibody, or by exposure to a chromogenic or fluorogenic substrate of the probe-attached enzyme. If the product contains biotin or, some other chemical group for which there are specific binding molecules, like avidin and antibodies, then the immobilized amplified product can be detected with an enzyme attached to the specific binding molecule, such as horseradish peroxidase or alkaline phosphatase attached to streptavidin.

VII. Kits In a further aspect of the present invention, kits suitable for use in carrying out the hybridization and detection methods of the present invention are provided. Such test kits, designed to facilitate the hybridization and detection of non-viral organisms, will generally comprise: a nucleic acid probe that is substantially complementary to a subsequence of SRP RNA from the group of non-viral organisms, which probe has the ability to hybridize to SRP from the group of non-viral organisms, but does not detectably hybridize to SRP RNA from other non-viral organisms that do not belong to the group. Optionally, the kit comprises one or more additional probes that are substantially complementary to a subsequence of SRP RNA and hybridize to the SRP RNA under stringent conditions. Optionally, the kit comprises a nucleic acid probe that is an adaptor probe, and a gel immobilized nucleic acid that is substantially complementary to a subsequence of the adaptor probe. The test kits can further comprise published instructions and reagents for detection of the targeted sequence.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill in the art will readily recognize a variety of noncritical parameters that could be changed or modified to yield essentially similar results.

### Example I

### Northern Blot of Bacterial Species With Conserved 4.5S 21-mer Probe

A probe based on a conserved region 22 nucleotide of bacteria 4.5S SRP RNA (see nucleotides 44-65 of E. coli 4.5S RNA) was used to probe a northern blot with RNA from a variety of bacterial species.

Total RNA was isolated from seven different organisms by standard methods. RNA concentration was measured using a spectrophotometer by UV absorbance (A260). RNA samples of 1 ig each were electrophoresed on a 5% polyacrylamide gel (29:1 acrylamide:bis) containing 8 M urea and 0.5×TBE (tris-borate EDTA) running buffer.

Samples contained 1× denaturing buffer (2× buffer=2×TBE, 13% ficoll w/v, 0.01% bromophenol blue, 0.05% xylene cyanol FF and 7M urea) in a total volume of 6 μl. Samples were heated to 80° C. for 2 minutes before loading. The gel was run for 1 hour at 120V at room temperature.

Following electrophoresis, the gel was stained with ethidium bromide and RNA bands were visualized by UV illumination. Under these conditions good separation of small RNAs was achieved. The gel was electroblotted onto a Hybond filter (Amersham) using a CBS Scientific blotting device according to manufacturer's instructions. The filter was baked at 80° C. for 2 hours, then hybridized to an oligonucleotide probe complementary to 4.5S RNA (see probe sequence, below). The probe was labeled with ³²P by polynucleotide kinase and [ã-³²P]ATP. This probe is a 41 mer with 21 nucleotides at the 5′ end which are complementary to the conserved 22 nucleotides found in the central region of 4.5S. The filters were hybridized in Rapidhyb (Amersham) for 1 hour at 42° C. and then washed twice in 5×SSC, 0.1% SDS for 5 minutes at room temperature, once in 0.5×SSC, 0.1% SDS at room temperature for 5 minutes, and finally twice in 0.5×SSC, 0.1% SDS for 10 minutes at 42° C.

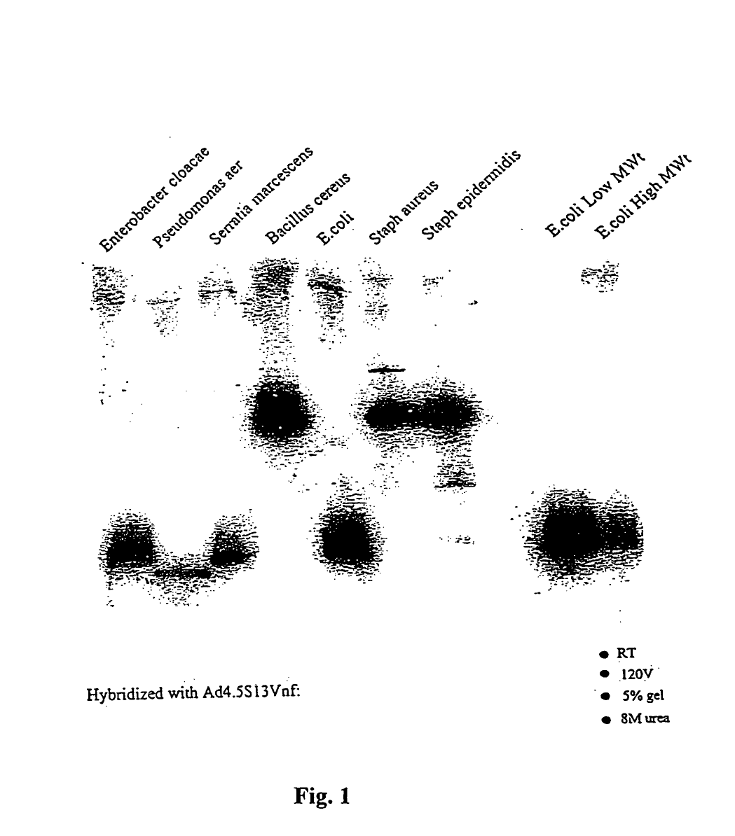

For detection of the labeled bands by autoradiography, the filters were wrapped in clear film (Saranwrap) and exposed to X-ray film at room temperature (see FIG. 1).

Sequence of Oligonucleotide Probe Used:

4.5S probe (Ad4.5): GCTGCTTCCTTCCGGACCTGAGTGAATACGTTCCCGGGCCT (SEQ ID NO:7)

(region underlined is complementary to 4.5S)

### Example II

### Estimation of Copy Number for 4.5S RNA in E. coli by Northern Blot

A probe to E. coli 4.5S RNA was used to estimate the copy number of 4.5S RNA, using 5S rRNA as a control.

Total RNA was isolated from a log phase culture of E. coli by standard methods. The RNA was treated with DNase 1, phenol/chloroform extracted, ethanol precipitated and resuspended in 1% SDS. Concentration was measured using a spectrophotometer by UV absorbance (A260). RNA samples were electrophoresed on a 5% polyacrylamide gel (29:1 acrylamide:bis) containing 8M urea and 0.5×TBE (tris-borate EDTA) running buffer. Samples containing increasing amounts of RNA were prepared (0.15 ig, 0.3 ig and 0.6 ig) in 1× denaturing buffer (2× buffer=2×TBE, 13% ficoll w/v, 0.01% bromophenol blue, 0.05% xylene cyanol FF and 7 M urea) in a total volume of 6 μl. Samples were heated to 80° C. for 2 minutes before loading. The gel was run for 1 hour at 100V at room temperature.

Following electrophoresis the gel was stained with ethidium bromide and RNA bands were visualized by UV illumination. Under these conditions, good separation of small RNAs was achieved. The gel was electroblotted onto a Hybond filter (Amersham) using a CBS Scientific blotting device according to manufacturer's instructions. The filter was baked at 80° C. for 2 hours and cut in half. One half of the filter was hybridized to an oligonucleotide probe complementary to 5S rRNA labeled with ³²P by polynucleotide kinase and [ã-³²P]ATP. Similarly, the other half of the filter (containing the same samples) was hybridized to a probe for E. coli 4.5S SRP RNA (probe 2nf). The filters were hybridized in Rapidhyb (Amersham) for 1 hour at 42° C. and then washed twice in 5×SSC, 0.1% SDS for 5 minutes at room temperature and 0.5×SSC, 0.1% SDS for 10 minutes at 42° C. The filters were wrapped in clear film (Saranwrap) and exposed to X-ray film at room temperature.

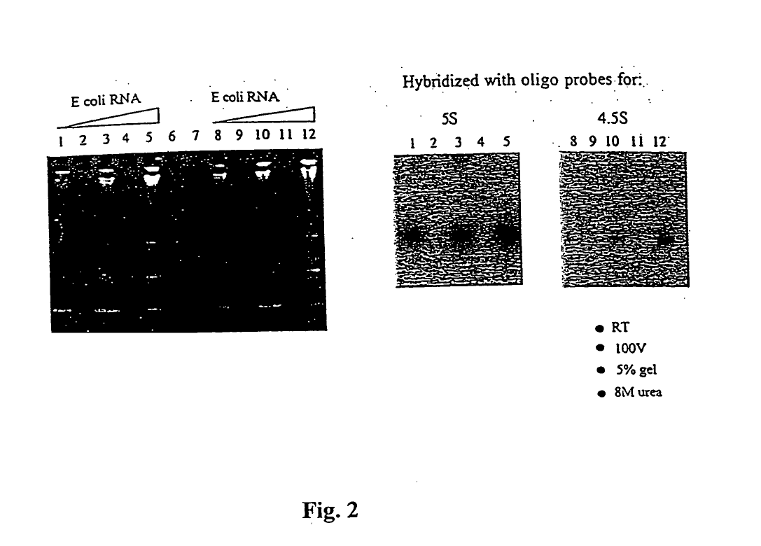

Bands corresponding to 5S rRNA and 4.5S SRP RNA were visualized by developing the X-ray film and cut out of the filters for Cherenkov counting. Relative abundance of the two RNA species was determined from the number of counts in the respective bands making adjustment for background counts and the relative specific activities of the two probes. From these calculations the 5S rRNA is 8.5 fold more abundant than 4.5S. If one assumes 18,700 copies of 5S RNA per cell (Neidhardt et al., in Escherichia coli and Salmonella typhimurium, Cellular and Molecular Biology, vol. 1, pp. 3-6 (Neidhardt et al., eds. 1987)); this gives the result that there are 2187 copies of 4.5S RNA per cell (see FIG. 2).

Sequences of Oligonucleotides Used:

| | | |
|---|---|---|
| 4.5S probe (2nf): | GGCACACGCGTCATCTGC | (SEQ ID NO:8) |
| 5S probe (66nf): | CCAGACTACCATCGGCGCT | (SEQ ID NO:9) |

### EXAMPLE III

### Detection of 4.5S RNA in a Culture of E. coli by Adapter-capture Following Sandwich Hybridization

A gel-immobilized probe was used to capture E. coli 4.5S RNA, which was first hybridized to an adaptor probe (complementary to the gel-immobilized probe) and a fluorescently labeled probe (see FIGS. 4-6).

0.5 ml of an overnight culture of E. coli was spun in a microfuge at 14 k for 20 seconds. Low molecular weight RNA was extracted using a Qiagen RNA/DNA kit according to manufacturer's instructions. Purified RNA was resuspended in 20 μl of RNase free water. 3 μl of this RNA was hybridized to two oligonucleotides: a fluorescent sandwich probe to label the target 4.5S RNA and an adapter capable of hybridizing to both the target RNA and to the capture acrydite oligo (see FIG. 5). The hybridization mix contained 100 mM NaCl, 0.5 pmoles of adapter (Ad4.5S13Vnf) and 5 pmoles of fluorescent sandwich probe (2F), in a total volume of 10 μl. The mix was placed on a heating block at 60° C., which was then switched off and allowed to cool to room temperature. 2 μl of ficoll loading buffer (35% ficoll 400, 0.1% xylene cyanol and bromophenol blue), was added to each hybridization mix and the whole sample was loaded on a 5% acrylamide gel containing a capture layer (29:1, acrylamide:bis and 0.5×TBE running buffer). The capture layer contained 10 μM acrydite capture oligonucleotide 13III-ac polymerized into the gel (see FIG. 5 and FIG. 6).

The gel is poured in three segments so that the middle segment of the gel forms the “capture layer.” The gel was prepared as follows: 10 ml of 5% polyacrylamide gel-mix containing 0.5×TBE was prepared by dilution from stock solutions (BioRad). 2 ml of the mix was removed to another tube for making the top segment of the gel. 80 μl of 10% ammonium persulphate solution and 8 μl of TEMED were added to the remaining 8 ml of gel-mix. 7.3 ml of this mix was poured into the gel mold to form the bottom segment of the gel. About 1 ml of 100% absolute ethanol was layered on the gel to give a level surface. After polymerization, the ethanol was removed and the gel was washed with water using a wash bottle. The last drops of water were removed from the surface of the gel.

The capture layer was made by mixing together 75 μl of 40% acrylamide stock solution (29:1 acrylamide:bis), 30 μl TBE, 535 μl deionized water, and 60 μl of 13III-ac acrydite DNA (100 μM). 6 μl of ammonium persulphate and 0.6 μl of TEMED were added and the mix was poured on top of the previously polymerized acrylamide. Again, 100% absolute ethanol was layered on top to give a sharp top surface with no meniscus. After polymerization, the ethanol was removed and the gel was washed with water as before. The top segment of the gel was then poured on top of the capture layer. The top segment contained 2 ml acrylamide, 20 μl ammonium persulphate, and 2 μl TEMED. A slot forming comb was inserted into the top segment and the gel was left for 5 minutes to polymerize.

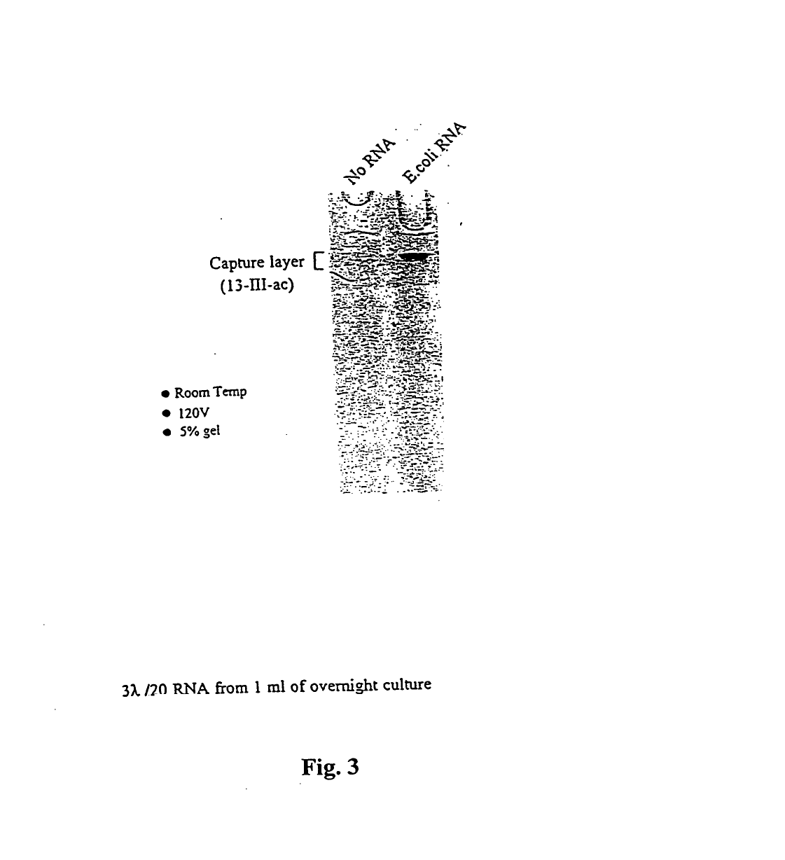

The gel was run at 120 volts for 1 hour, 20 minutes. The image was visualized and analyzed on a Molecular Dynamics Fluorimager 595 (see FIG. 3).

Sequences of Oligonucleotides Used:

| | | | |
|---|---|---|---|
| Acrydite capture probe: | 13-III-ac: | AC-TTTTTTTTTAGGCCCGGGAACGTATTCAC | (SEQ ID NO:12) |
| Fluorescent sandwich probe | 2F: | GGCACACGCGTCATCTGC | (SEQ ID NO:13) |
| Adapter: | Ad4.5S13V: | GCTGCTTCCTTCCGGACCTGAGTGAATACGTTCCCGGGCCT | (SEQ ID NO:7) |

### Example IV

### Detection of Different Bacterial 4.5S RNAs Using Pooled Probes

A pool of five gel-immobilized probes were used to capture 4.5S RNAs from nine bacterial species, which had first been hybridized to a pool of two reporter probes conjugated to alkaline phosphatase.

Exponentially growing bacteria were chilled on ice and aliquots were diluted and spread on agar plates to count the number of colony forming units (cfu). Aliquots of 10 μl volumes of the cultures were frozen at −70° C. The total numbers of bacteria in an aliquot for each species are given in the following table.

TABLE A
Numbers of Bacteria Detected

| Bacterium | cfu/aliguot |
|---|---|
| Escherichia ccli | 1.5 × 10⁷ |
| Bacilllus cereus | 6.7 × 10⁶ |
| Enterobacter cloacae | 6.0 × 10⁷ |
| Klebsiella pneumoniae | 4.9 × 10⁷ |
| Pseudomonas aeruginosa | 2.4 × 10⁶ |
| Serratia marcescens | 4.8 × 10⁷ |
| Staphylococcus aureus | 2.1 × 10⁷ |
| Staphylococcus epidermidis | 2.2 × 10⁷ |
| Staphylococcus warneri | 5.5 × 10⁶ |

Aliquots were thawed, 20% sodium dodecyl sulfate was added to a final concentration of 1.4% in a total volume of 15.6 μl, and tubes were heated at 130° C. for 10 minutes. Tubes were removed to room temperature for several minutes, and hybridization mix was added to a final volume of 20 pl with the following final concentrations: 120 mM NaCl, 1 mM MgCl₂, 0.1 mM ZnCl₂, 22.5 mM Tris (pH 8), 22.5 mM boric acid, 0.5 mM aurin tricarboxylic acid, 8 mM Na phosphate, and 50 nM of each of the alkaline phosphate-conjugated reporter probes, RP-1 (5′-alkaline phosphatase-GCUGCUUCCUUC (SEQ ID NO:4); underlined bases represent 2′-O-methyl RNA nucleotides) and RP-2 (5′-alkaline phosphatase-GCUGCUUCCGUC (SEQ ID NO: 14). These mixtures were warmed to 550C for 10 minutes, then removed to room temperature and 4 μl of loading buffer (50% glycerol, 0.2% xylene cyanole, 0.2% bromphenol blue) added. Half of each mixture was loaded onto a 5% polyacrylamide gel (89 mM Tris (pH 8.5), 27 mM phosphate buffer), made with 10 μM of each of the following five acrydite-modified, 2′-O-methyl RNA capture probes, polymerized into the gel in a fashion similar to that described in Example III.

| | |
|---|---|
| CP-1 | |
| 5′-acrydite-TTTTTT-CGGACCUGACCUG | (SEQ ID NO:15) |
| CP-2 | |
| 5′-acrydite-TTTTTT-AGGACCUGACAUG | (SEQ ID NO:16) |
| CP-3 | |
| 5′-acrydite-TTTTTT-CGGACCUGACCAG | (SEQ ID NO:17) |
| CP-4 | |
| 5′-acrydite-TTTTTT-CGGACCUGACAAG | (SEQ ID NO:18) |
| CP-5 | |
| 5′-acrydite-TTTTTT-CGGAUCUGACACG | (SEQ ID NO:19) |

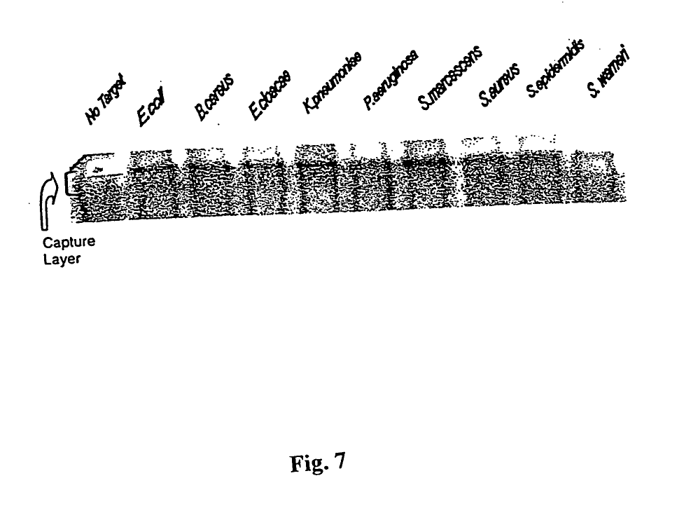

The gel was run at 30° C. at 20 volts/cm for 30 minutes, rinsed in diethanolamine buffer (2.4 M diethanolamine, 1 mM MgCl₂, 0.1 mM ZnCl₂, pH 10) for 10 minutes, then AttoPhoS™ chemifluorescent substrate (Boehringer-Mannheim) was added for 10 minutes. The reaction was stopped by the addition of 1 M Na phosphate (pH 7.2) and the fluorescent signal was scanned on a Molecular Dynamics Fluorimager 595 (see FIG. 7). All nine of the listed bacterial species were detected with this probe set.

What is claimed is:

1. A method for detecting in a sample a non-viral organism belonging to a group, the group consisting of at least one, but less than all non-viral organisms, the method comprising the steps of:
(i) contacting a sample comprising SRP RNA with a nucleic acid probe, wherein the nucleic acid probe is substantially complementary to a subsequence of SRP RNA from the group of non-viral organisms;
(ii) incubating the sample comprising SRP RNA and the nucleic acid probe under hybridization conditions such that the nucleic acid probe hybridizes to SRP RNA from the group of non-viral organisms but does not detectably hybridize to SRP RNA from other non-viral organisms that do not belong to the group; and,
(iii) detecting hybridization of the nucleic acid probe to SRP RNA.

2. The method of claim 1, wherein the nucleic acid probe comprises a detectable moiety.

3. The method of claim 1, wherein the SRP RNA comprises a detectable moiety.

4. The method of claim 1; wherein the step of contacting further comprises the use of one or more additional nucleic acid probes that are substantially complementary to a subsequence of SRP RNA from the non-viral organism and have the ability to hybridize under stringent conditions to the SRP RNA from the non-viral organism.

5. The method of claim 4, wherein one of the nucleic acid probes comprises a detectable moiety.

6. The method of claim 1, wherein the nucleic acid probe is about 8 to about 50 nucleotides in length.

7. The method of claim 1, wherein the nucleic acid probe is about 15 to about 25 nucleotides in length.

8. The method of claim 1, wherein the nucleic acid probe is selected from the group consisting of DNA, PNA, and 2′-O-methyl RNA.

9. The method of claim 1, wherein the nucleic acid probe is a PNA.

10. The method of claim 1, wherein the nucleic acid probe is perfectly complementary to the subsequence of SRP RNA.

11. The method of claim 1, wherein the SRP RNA is 4.5S RNA.

12. The method of claim 1, wherein the sample is from a human.

13. The method of claim 1, wherein the non-viral organism belonging to the group is a fungus.

14. The method of claim 13, wherein the fungus is selected from the group consisting of Candida sp., Cryptococcus sp., Aspergillus sp., Histoplasma sp., and Microsporum sp.

15. The method of claim 1, wherein the non-viral organism belonging to the group is a protozoan.

16. The method of claim 15, wherein the protozoan is selected from the group consisting of Pneumocystis sp., Toxoplasma sp., Cryptosporidium sp., Giardia sp., Leshmania sp., Trypanosoma sp., Plasmodium sp., Acanthamoeba sp., and Entamoeba sp.

17. The method of claim 1, wherein the non-viral organism belonging to the group is a bacterium.

18. The method of claim 17, wherein the bacterium is selected from the group consisting of Propionibacterium sp., Klebsiella sp., Enterobacter sp., Serratia sp., Salmonella sp., Legionella sp., Pseudomonas sp., Haemophilus sp., Escherichia sp., Mycoplasma sp., Micrococcus sp., Listeria sp., Bacillus sp., Staphylococcus sp., Streptococcus sp., Clostridia sp., Neisseria sp., Helicobacter sp., Vibrio sp., Campylobacter sp., Bordetella sp., Ureaplasma sp., Treponema sp., Leptospira sp., Borrelia sp., Actinomyces sp., Nocardia sp., Chlamydia sp., Rickettsia sp., Coxiella sp., Ehrilichia sp., Rochalimaea sp., Brucella sp., Yersinia sp., Fracisella sp., and Pasteurella sp.

19. The method of claim 1, wherein the nucleic acid probe has a nucleotide sequence selected from the group consisting of:
(SEQ ID NO:2)
GGTGCTTCGTTCCGGACCTGAC;
(SEQ ID NO:3)
GCTGCTTCCTTCCGGACCTGA;
(SEQ ID NO:9)
GGCACACGCGTCATCTGC;
(SEQ ID NO:4)
GCTGCTTCGTTC;
(SEQ ID NO:7)
GGTGCTTCCTTCCGGACCTGAGTGAATACGTTCCCGGGCCT;
(SEQ ID NO:8)
GCTGCTTCCTTCCGGACCTGACAAAAACGATAAACCAACCA;
(SEQ ID NO:11)
GCTGCTTCCTTCCGGACCTGACCTGGTAAA;
(SEQ ID NO:5)
GCTGCTTCCTTCCG;
(SEQ ID NO:6)
GACCTGACCTGGTA;
(SEQ ID NO:14)
GCTGCTTCCGTC;
(SEQ ID NO:15)
CGGACCTGACCTG;
(SEQ ID NO:16)
AGGACCUGACAUG;
(SEQ ID NO:17)
CGGACCUGACCAG;
(SEQ ID NO:18)
CGGACCUGACAAG; and
(SEQ ID NO:19)
CGGAUCUGACACG.

20. A method for detecting in a sample a non-viral organism belonging to a group, the group consisting of at least one, but less than all of non-viral organisms, the method comprising the steps of:
(i) contacting a sample comprising SRP RNA with a nucleic acid probe, wherein the nucleic acid probe is substantially complementary to a subsequence of SRP RNA from the group of non-viral organisms and wherein the nucleic acid probe has the ability to hybridize under stringent conditions to the SRP RNA from the group of non-viral organisms;
(ii) incubating the sample comprising SRP RNA and the nucleic acid probe under stringent hybridization conditions to form duplex SRP RNA from the group of non-viral organisms;
(iii) contacting the duplex SRP RNA with a gel-immobilized nucleic acid probe, wherein the gel-immobilized nucleic acid probe is substantially complementary to a subsequence of the duplex SRP RNA from the group of non-viral organisms;
(iv) incubating the duplex SRP RNA and the gel-immobilized nucleic acid probe under hybridization conditions such that the gel-immobilized nucleic acid probe hybridizes to a subsequence of the duplex SRP RNA from the group of organisms, but does not detectably hybridize to SRP RNA from other non-viral organisms that do not belong to the group; and,
(v) detecting hybridization of the gel-immobilized probe to duplex SRP RNA.

21. The method of claim 20, wherein step (iv) further comprises electrophoresing the sample comprising duplex SRP RNA through a gel.

22. The method of claim 20, wherein the nucleic acid probe comprises a detectable moiety.

23. The method of claim 20, wherein the SRP RNA comprises a detectable moiety.

24. The method of claim 20, wherein the step of contacting with a nucleic acid probe further comprises the use of one or more additional nucleic acid probes.

25. The method of claim 24, wherein one of the nucleic acid probes comprises a detectable moiety.

26. The method of claim 20, wherein the nucleic acid probe is an adaptor probe comprising a subsequence that hybridizes under stringent conditions to the gel25 immobilized probe.

27. The method of claim 20, wherein the gel-immobilized nucleic acid probe and the nucleic acid probe each comprise a subsequence that is substantially complementary to the same SRP RNA subsequence.

28. The method of claim 20, wherein the gel-immobilized nucleic acid probe and the nucleic acid probe are about 8 to about 50 nucleotides in length.

29. The method of claim 20, wherein the nucleic acid probe is about 15 to about 25 nucleotides in length.

30. The method of claim 20, wherein the gel-immobilized nucleic acid probe and the nucleic acid probe are selected from the group consisting of DNA, PNA, and 2-O-methyl RNA.

31. The method of claim 20, wherein the gel-immobilized nucleic acid probe and the nucleic acid probe are PNA.

32. The method of claim 20, wherein the gel-immobilized nucleic acid probe is perfectly complementary to the subsequence of SRP RNA.

33. The method of claim 20, wherein the SRP RNA is 4.5S RNA.

34. The method of claim 20, wherein the sample is from a human.

35. The method of claim 20, wherein the non-viral organism belonging to the group is a fungus.

36. The method of claim 35, wherein the fungus is selected from the group consisting of Candida sp., Cryptococcus sp., Aspergillus sp., Histoplasma sp., and Microsporum sp.

37. The method of claim 20, wherein the non-viral organism belonging to the group is a protozoan.

38. The method of claim 37, wherein the protozoan is selected from the group consisting of Pneumocystis sp., Toxoplasma sp., Cryptosporidium sp., Giardia sp., Leshmania sp., Trypanosoma sp., Plasmodium sp., Acanthamoeba sp., and Entamoeba sp.

39. The method of claim 20, wherein the non-viral organism belonging to the group is a bacterium.

40. The method of claim 39, wherein the bacterium is selected from the group consisting of Propionibacterium sp., Klebsiella sp., Enterobacter sp., Serratia sp., Salmonella sp., Legionella sp., Pseudomonas sp., Haemophilus sp., Escherichia sp., Mycoplasma sp., Micrococcus sp., Listeria sp., Bacillus sp., Staphylococcus sp., Streptococcus sp., Clostridia sp., Neisseria sp., Helicobacter sp., Vibrio sp., Campylobacter sp., Bordetella sp., Ureaplasma sp., Treponema sp., Leptospira sp., Borrelia sp., Actinomyces sp., Nocardia sp., Chlamydia sp., Rickettsia sp., Coxiella sp., Ehrilichia sp., Rochalimaea sp., Brucella sp., Yersinia sp., Fracisella sp., and Pasteurella sp.

41. The method of claim 20, wherein the gel-immobilized nucleic acid probe has the nucleotide sequence selected from the group consisting of:

| | |
|---|---|
| GCTGCTTCCTTCCGGACCTGAC; | (SEQ ID NO:2) |
| GCTGCTTCCTTCCGGACCTGA; | (SEQ ID NO:3) |
| GGCACACGCGTCATCTGC; | (SEQ ID NO:9) |
| GCTGCTTCCTTC; | (SEQ ID NO:4) |
| GCTGCTTCCTTCCGGACCTGACCTGGTAAA; | (SEQ ID NO:11) |
| GCTGCTTCCTTCCG; | (SEQ ID NO:5) |
| GACCTGACCTGGTA; | (SEQ ID NO:6) |
| GCTGCTTCCGTC; | (SEQ ID NO:14) |
| CGGACCTGACCTG; | (SEQ ID NO:15) |
| AGGACCUGACAUG; | (SEQ ID NO:16) |
| CGGACCUGACCAG; | (SEQ ID NO:17) |
| CGGACCUGACAAG; and | (SEQ ID NO:18) |
| CGGAUCUGACACG. | (SEQ ID NO:19) |

42. The method of claim 20, wherein the nucleic acid probe has a nucleotide sequence selected from the group consisting of:
(SEQ ID NO:7)
GCTGCTTCCTTCCGGACCTGAGTGAATACGTTCCCGGGCCT; and
(SEQ ID NO:8)
GCTGCTTCCTTCCGGACCTGACAAAAACGATAAACCAACCA.

43. The method of claim 26, wherein the adaptor has a nucleotide sequence selected from the group consisting of:
(SEQ ID NO:7)
GCTGCTTCCTTCCGGACCTGAGTGAATACGTTCCCGGGCCT; and
(SEQ ID NO:8)
GCTGCTTCCTTCCGGACCTGACAAAAACGATAAACCAACCA.

44. A kit for detecting in a sample a non-viral organism belonging to a group, the group consisting of at least one, but less than all non-viral organisms, said kit comprising a container comprising a nucleic acid probe that is substantially complementary to a subsequence of SRP RNA from the group of non-viral organisms, wherein the nucleic acid probe has the ability to hybridize to SRP RNA from the group of non-viral organisms, but does not detectably hybridize to SRP RNA from other non-viral organisms that do not belong to the group.

45. The kit of claim 45, wherein the nucleic acid probe comprises a detectable moiety.

46. The kit of claim 45, further comprising one or more additional nucleic acid probes that are substantially complementary to a subsequence of SRP RNA from the non-viral organism and have the ability to hybridize under stringent conditions to the SRP RNA from the non-viral organism.

47. The kit of claim 45, wherein one of the nucleic acid probes comprises a detectable moiety.

48. The kit of claim 45, wherein the nucleic acid probe is an adaptor probe.

49. The kit of claim 45, wherein the nucleic acid probe is a gel-immobilized nucleic acid probe.

50. The kit of claim 45, further comprising a gel-immobilized nucleic acid probe, wherein the gel-immobilized probe is substantially complementary to a subsequence of the adaptor probe and hybridizes under stringent conditions to the adaptor probe.


SEQUENCE LISTING (27 sequences)

SEQ ID NO: 1 - RNA, 21 nt, Escherichia coli
gucagguccggaaggaagcag

SEQ ID NO: 2 - DNA, 22 bp, Artificial Sequence
  Complementary sequence of conserved E. coli 4.5S RNA region nucleotide 44-65
gctgcttccttccggacctgac

SEQ ID NO: 3 - DNA, 21 bp, Artificial Sequence
  Description of Artificial Sequence shortened probe derived from complementary sequence of E. coli 4.5S RNA region 44-65
gctgcttccttccggacctga

SEQ ID NO: 4 - DNA, 12 bp, Artificial Sequence
  Description of Artificial Sequencea shorter probe derived from complementary sequence of E. coli 4.5S RNA region 44-65
gctgcttccttc

SEQ ID NO: 5 - DNA, 14 bp, Artificial Sequence
  Description of Artificial Sequence a short probe derived from complementary sequence of E. coli 4.5S RNA region 44-65
gctgcttccttccg

SEQ ID NO: 6 - DNA, 14 bp, Artificial Sequence
  Description of Artificial Sequence a short probe derived from the complementary sequence of E. coli 4.5S RNA region 44-65
gacctgacctggta

SEQ ID NO: 7 - DNA, 41 bp, Artificial Sequence
  Description of Artificial Sequenceadaptor probe
gctgcttccttccggacctgagtgaatacgttcccgggcct

SEQ ID NO: 8 - DNA, 41 bp, Artificial Sequence
  Description of Artificial Sequenceadaptor probe
gctgcttccttccggacctgacaaaaacgataaaccaacca

SEQ ID NO: 9 - DNA, 18 bp, Artificial Sequence
  Description of Artificial Sequenceadaptor probe
ggcacacgcgtcatctgc

SEQ ID NO: 10 - RNA, 29 nt, Escherichia coli
uuuaccaggucagguccggaaggaagcag

SEQ ID NO: 11 - DNA, 30 bp, Artificial Sequence
  Description of Artificial Sequence complementary sequence of conserved E. coli 4.5S RNA region nucleotides 36-65
gctgcttccttccggacctgacctggtaaa

SEQ ID NO: 12 - DNA, 29 bp, Artificial Sequence
  Description of Artificial Sequence acrydite capture probe 13-III-ac
tttttttttaggcccgggaacgtattcac

SEQ ID NO: 13 - DNA, 18 bp, Artificial Sequence
  Description of Artificial Sequencefluorescent sandwich probe 2F
ggcacacgcgtcatctgc

SEQ ID NO: 14 - DNA, 12 bp, Artificial Sequence
  Description of Artificial Sequencealkaline phosphatase conjugated reporter probe RP-2
nnnnnnnnnnnn

SEQ ID NO: 15 - DNA, 19 bp, Artificial Sequence
  Description of Artificial Sequencegel- immobilized acrydite-modified capture probe CP-1
ttttttnnnnnnnnnnnnn

SEQ ID NO: 16 - DNA, 19 bp, Artificial Sequence
  Description of Artificial Sequencegel- immobilized acrydite-modified capture probe CP-2
ttttttnnnnnnnnnnnnn

SEQ ID NO: 17 - DNA, 19 bp, Artificial Sequence
  Description of Artificial Sequencegel- immobilized acrydite-modified capture probe CP-3
ttttttnnnnnnnnnnnnn

SEQ ID NO: 18 - DNA, 19 bp, Artificial Sequence
  Description of Artificial Sequencegel- immobilized acrydite-modified capture probe CP-4
ttttttnnnnnnnnnnnnn

SEQ ID NO: 19 - DNA, 19 bp, Artificial Sequence
  Description of Artificial Sequencegel- immobilized acrydite-modified capture probe CP-5
ttttttnnnnnnnnnnnnn

SEQ ID NO: 20 - DNA, 19 bp, Artificial Sequence
  Description of Artificial Sequence 5S probe (66nf)
ccacactaccatcggcgct

SEQ ID NO: 21 - DNA, 12 bp, Artificial Sequence
  Description of Artificial Sequence nucleic acid probe
gctgcttccgtc

SEQ ID NO: 22 - DNA, 13 bp, Artificial Sequence
  Description of Artificial Sequence nucleic acid probe
cggacctgacctg

SEQ ID NO: 23 - RNA, 13 nt, Artificial Sequence
  Description of Artificial Sequence nucleic acid probe
aggaccugacaug

SEQ ID NO: 24 - RNA, 13 nt, Artificial Sequence
  Description of Artificial Sequence nucleic acid probe
cggaccugaccag

SEQ ID NO: 25 - RNA, 13 nt, Artificial Sequence
  Description of Artificial Sequence nucleic acid probe
cggaccugacaag

SEQ ID NO: 26 - RNA, 13 nt, Artificial Sequence
  Description of Artificial Sequence nucleic acid probe
cggaucugacacg

SEQ ID NO: 27 - RNA, 12 nt, Artificial Sequence
  Description of Artificial Sequencealkaline phosphatase-cojugated reporter probe RP-1
nnnnnnnnnnnn